(19) United States
(12) Patent Application Publication
(10) Pub. No.: US 2024/0057611 A1
(43) Pub. Date: Feb. 22, 2024

(54) MICROBIAL MATERIAL, PLANT CULTIVATION METHOD, AND BACTERIAL STRAIN

(71) Applicant: TOHOKU UNIVERSITY, Miyagi (JP)

(72) Inventors: Kiwamu MINAMISAWA, Miyagi (JP); Sawa HARA, Miyagi (JP); Manabu ITAKURA, Miyagi (JP); Arthur FERNANDES SIQUEIRA, Miyagi (JP)

(21) Appl. No.: 18/260,601

(22) PCT Filed: Jan. 6, 2022
(86) PCT No.: PCT/JP2022/000178

§ 371 Date: Jul. 6, 2023

(30) Foreign Application Priority Data
Jan. 9, 2021 (JP) 2021-002474

(51) Int. Cl. A01N 63/20; C12N 1/20; A01G 7/06

(52) U.S. Cl. CPC A01N 63/20; C12N 1/205; A01G 7/06; C12R 2001/01

(57) ABSTRACT

An object is to provide a bacterial strain having superior nitrogen fixing capability and N₂O reduction capability and a microbial material containing the bacterial strain. The object can be achieved by a microbial material containing a bacterial strain belonging to the Bradyrhizobium ottawaense Glade having a nitrogen fixing capability and an N₂O reduction capability.

### TECHNICAL FIELD

The disclosure in this application relates to a microbial material, a plant cultivation method, and a bacterial strain.

### BACKGROUND ART

Bradyrhizobium soybean rhizobia are plant symbiotic microorganisms that live symbiotically with roots of soybeans, form root nodules thereon, and fix nitrogen contained in the atmosphere, and most of which also possess a denitrification capability. Among others, the B. diazoefficiens possesses a N₂O reductase gene (nos) that reduces N₂O into N₂, which is the final reaction of denitrification. N₂O produced in a denitrification process exhibits a greenhouse effect about 300 times greater than CO₂, and suppression thereof is an important challenge. Inoculation of B. diazoefficiens possessing N₂O reductase into a soybean farm field can suppress N₂O generation from a soybean rhizosphere. Further, it is known that N₂O reduction activity of the B. diazoefficiens is enhanced by gene recombination technologies (see Non-Patent Literatures 1 and 2).

### CITATION LIST

### Non-Patent Literature

Non-Patent Literature 1: K. Minamisawa et al., “Regulation of nitrous oxide reductase genes by NasT-mediated transcription antitermination in Bradyrhizobium diazoefficiens”, Environ Microbiol Reports, 2017, 9(4), p389-396
Non-Patent Literature 2: K. Minamisawa et al., “The nitrate-sensing NasST system regulates nitrousoxide reductase and periplasmic nitrate reductase in Bradyrhizobium japonicum”, Environmental Microbiology, 2014, doi: 10. 1111/1462-2920. 12546

### SUMMARY OF INVENTION

### Technical Problem

The nos-enhanced mutant strains disclosed in Non-Patent Literatures 1 and 2 have a stronger N₂O reduction capability than wild-type strains. However, an international framework has been established for use of genetically modified organisms or the like in order to prevent adverse effects on biodiversity. Similarly, in Japan, regulatory measures have been taken for use of genetically modified organisms or the like in accordance with “Act on the Conservation and Sustainable Use of Biological Diversity through Regulations on the Use of Living Modified Organisms” (known as the Cartagena Act). Therefore, there is a problem that the nos-enhanced mutant strains disclosed in Non-Patent Literatures 1 and 2 are not allowed to use in the natural environment. It is thus desired to discover a bacterial strain that has a nitrogen fixing capability and thus can promote growth of plants and further has a superior N₂O reduction capability.

The disclosure of this application has been made to solve the above problem and, through an intensive study, has newly found that bacterial strains superior in the nitrogen fixing capability and the N₂O reduction capability exist in the Bradyrhizobium ottawaense and such bacterial strains are useful for a microbial material.

That is, an object of the disclosure of this application is to provide a microbial material, a plant cultivation method, and a bacterial strain.

### Solution to Problem

(1) A microbial material including a bacterial strain belonging to a Bradyrhizobium ottawaense Glade having a nitrogen fixing capability and an N₂O reduction capability.
(2) The microbial material according to (1) described above, in which the bacterial strain belongs to a Glade including a B. ottawaense type strain OO99 in evolutionary phylogenetic analysis in which the B. ottawaense type strain OO99 and one or more Bradyrhizobium distinct species thereof are included in an operational taxonomic unit (OTU).
(3) The microbial material according to (2) described above, in which the evolutionary phylogenetic analysis creates and analyzes, for each OTU, a linked sequence in which amino acid sequences are linked to each other, the amino acid sequences being encoded by genes of dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, and tsf extracted by using AMPHORA.
(4) The microbial material according to (1) or (2) described above, in which the bacterial strain further has an ANI value for the B. ottawaense type strain OO99 that is 95% or higher in ANI analysis.
(5) The microbial material according to any one of claims (1), (2), and (4) described above, in which the bacterial strain further has an ITS base sequence having 97% or higher homology with an ITS (16S-23S rRNA intergenic region) base sequence of a Bradyrhizobium bacterium.
(6) The microbial material according to any one of (1) to (5) described above, in which the bacterial strain is SG09 (accession number: NITE BP-03361).
(7) The microbial material according to any one of (1) to (5) described above, in which the bacterial strain is SF21 (accession number: NITE BP-03552) or SH12 (accession number: NITE BP-03553).
(8) The microbial material according to any one of (1) to (7) described above that functions as a growth-promoting agent for a plant.
(9) The microbial material according to (8) described above, in which the plant is a leguminous plant.
(10) A plant cultivation method including a step of causing the microbial material according to any one of (1) to (9) described above to be in contact with a seed or a root of a plant or to be present near a root of a plant.
(11) A bacterial strain that belongs to a Bradyrhizobium ottawaense Glade having a nitrogen fixing capability and an N₂O reduction capability and belongs to a Glade including a B. ottawaense type strain OO99 in evolutionary phylogenetic analysis in which the B. ottawaense type strain OO99 and one or more Bradyrhizobium distinct species thereof are included in an operational taxonomic unit (OTU).
(12) The bacterial strain according to (11) described above, in which the bacterial strain is SG09 (accession number: NITE BP-03361).
(13) The bacterial strain according to (11) described above, in which the bacterial strain is SF21 (accession number: NITE BP-03552) or SH12 (accession number: NITE BP-03553).

### Advantageous Effect

The microbial material disclosed in this application has a nitrogen fixing capability and a N₂O reduction capability. It is therefore possible to promote growth of a plant and suppress emission of N₂O that is a greenhouse gas.

### BRIEF DESCRIPTION OF THE DRAWINGS

FIG. 1A is a diagram illustrating an evolutionary phylogenetic tree by AMPHORA (housekeeping gene) of Bradyrhizobium bacterial strains, and FIG. 1B is a diagram illustrating the function of each bacterial strain.

FIG. 2 is a graph illustrating a survey of the occupancy rate of root nodules formed in Example 1.

FIG. 3 represents photographs substitute for a drawing, FIG. 3A is a photograph of soybeans grown in Example 2, and FIG. 3B is a photograph of soybeans grown in Comparative example 1.

FIG. 4 is a graph illustrating the difference in the N₂O reduction capability between wild-type strains belonging to the B. ottawaense Glade, a wild-type strain belonging to the B. diazoefficiens Glade, and mutant strains with an enhanced N₂O reduction capability.

FIG. 5A is a diagram illustrating an evolutionary phylogenetic tree by AMPHORA (housekeeping gene) of a Bradyrhizobium bacterial strain, and FIG. 5B is a diagram illustrating the function of each bacterial strain.

FIG. 6 is a photograph substitute for a drawing, which is a photograph of soybeans grown in Example 3 (SG09, SF21, SH12), Comparative example 2 (USDA110), and Comparative example 3 (no inoculation).

FIG. 7 illustrates the dry weight of root nodules (Nodules Dry Weight) of the soybeans grown in Example 3 and Comparative example 2 and the number of root nodules (Nodules Number) of the soybeans grown in Example 3 and Comparative example 2.

FIG. 8 is a graph illustrating the difference in the N₂O reduction capability between a wild-type strain belonging to the B. ottawaense Glade and a wild-type strain belonging to the B. diazoefficiens Glade.

FIG. 9 is a graph illustrating N₂O flux measurements of Example 4 (SG09) and Comparative example 5 (110ΔH1) relatively when the N₂O flux measurement of Comparative example 4 (USDΔ110) is defined as 1.

### DETAILED DESCRIPTION OF EMBODIMENTS

A microbial material and a plant cultivation method disclosed in this application will be described below in detail. Note that, in this specification, denotations of numerical values, abbreviations, and the like are understood as follows.

(1) A numerical range expressed by using “to” means a range including the numerical values written before and after the “to” as the lower limit value and the upper limit value.
(2) A numerical value, a numerical range, and a qualitative expression (for example, an expression such as “identical” or “same”) refer to a numerical value, a numerical range, and a nature including an error generally tolerated in this technical field.

### Embodiment of Microbial Material

The microbial material disclosed in this application includes a bacterial strain belonging to the Bradyrhizobium ottawaense Glade having a nitrogen fixing capability and a N₂O reduction capability (hereafter, which may be simply referred to as “bacterial strain”). Note that, in this specification, “microbial material” means a microbial (bacterial strain) itself inoculated into soil or a crop seed for the purpose of improvement on growth or health of a plant or means what is obtained by adding another component to the microbial.

A new bacterial strain disclosed in this application is isolated from a sorghum root of a farm field in Nihon-matsu city, Fukushima prefecture and belongs to the Bradyrhizobium. In the Bradyrhizobium, the diazoefficiens Glade having a N₂O reduction capability and the japonicum Glade having no N₂O reduction capability are known. On the other hand, the new bacterial strain disclosed in this application is included in the B. ottawaense Glade that is a superior soybean rhizobium isolated in Canada. As a result of survey on activities of many bacterial strains, the inventors have found that there are a bacterial strain having a N₂O reduction capability and a nitrogen fixing capability and a bacterial strain having a N₂O reduction capability but no nitrogen fixing capability in the ottawaense Glade. Therefore, the use of the bacterial strain belonging to the ottawaense Glade and having a nitrogen fixing capability and a N₂O reduction capability as a microbial material can promote growth of plants and reduce the amount of N₂O emission from a farm field. Note that, in this specification, “nitrogen fixing capability” means a capability of converting stable N₂ contained in the atmosphere into another nitrogen compound such as highly reactive NH₃, for example. Further, “N₂O reduction capability” means a capability of reducing N₂O, which is generated in a nitrification and denitrification process of NH₃, into N₂. Further, a bacterial strain “having a nitrogen fixing capability and a N₂O reduction capability” means having a gene that achieves the above capabilities.

The bacterial strains disclosed in this application belong to the ottawaense Glade and are not particularly limited if they have a nitrogen fixing capability and a N₂O reduction capability. As described later, it is only required to measure whether or not the nitrogen fixing capability and the N₂O reduction capability are present among screened bacterial strains.

A type strain of the B. ottawaense Glade is 0099 collected in Canada (Xiumei Yu et al., “Bradyrhizobium ottawaense sp. nov., a symbiotic nitrogen fixing bacterium from root nodules of soybeans in Canada”, International Journal of Systematic and Evolutionary Microbiology (2014), 64, 3202-3207).

As with Examples described later, the OO99 that is the B. ottawaense type strain is a wild type but has a superior N₂O reduction capability. The bacterial strain disclosed in this application belongs to a Glade including the B. ottawaense type strain OO99 in evolutionary phylogenetic analysis in which the B. ottawaense type strain OO99 and one or more Bradyrhizobium distinct species thereof are included in an operational taxonomic unit (OTU).

Note that the OTU described above is a unit obtained when base sequences of essential genes of bacteria (in general, 16S ribosomal RNA genes) are classified by similarity as an index on a computer. If the OTU is the same, it can be said that the bacteria are composed of the same evolving bacterial species.

The evolutionary phylogenetic analysis is performed by creating and analyzing linked sequences for each OUT, and the linked sequences are linked amino acid sequences encoded by dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, and tsf genes extracted by using AMPHORA.

The details of AMPHORA are described in (1) Martin Wu et al., “A simple, fast, and accurate method of phylogenomic inference”, Genome Biology 2008, 9: R151, and (2) Martin Wu et al., “Phylogenomic analysis of bacterial and archaeal sequences with AMPHORA2”, BIOINFORMATICS APPLICATIONS NOTE, vol. 28, no. 7, 2012, p1033-1034 (hereafter, denoted as “Wu and Scott, 2012”).

The bacterial strain may further have an ANI value for the B. ottawaense type strain OO99 that is 95% or higher in Average Nucleotide Identity (ANI) analysis. When the ANI value is 95% or higher, it can be determined that the bacterial strains are of the same species. Note that the ANI analysis can be performed by a known method.

The bacterial strain may further have an ITS base sequence having 97% or higher homology with an ITS (16S-23S rRNA intergenic region) base sequence of a Bradyrhizobium bacterium. The ITS is a region used for molecular phylogenetic analysis, and when the regions have 97% or higher homology, it can be said that the bacterial strains are of the same species.

The bacterial strain may be, for example, SG09 SG11, SF21, SH12, or the like but is not limited thereto.

Among the bacterial strains disclosed in this application, the SG09 was accepted at National Institute of Technology and Evaluation (NITE) Patent Microorganisms Depositary on Jan. 5, 2021, and the accession number thereof is “NITE P-03361”. The SG09 was transferred to NITE Patent Microorganisms Depositary as the international deposition under the Budapest Treaty, and was accepted on Nov. 8, 2021. The accession number thereof is “NITE BP-03361”.

The “SF21” and “SH12” were accepted at NITE Patent Microorganisms Depositary on Nov. 8, 2021, as the international deposition under the Budapest Treaty. The accession numbers thereof are as below.
“SF21”: “NITE BP-03552”.
“SH12”: “NITE BP-03553”.

Any known culture media can be used for culturing the bacterial strain disclosed in this application. Further, other than liquid culture media, a solid culture medium such as a slope culture medium and a plate culture medium with agar can also be used. The use of these culture media makes it possible to proliferate bacterial strains to obtain a desired number of bacterial cells.

Any source with which the above bacterial strains may be assimilated can be used as the carbon source of a culture medium, which may be, for example, a sugar such as glucose, galactose, lactose, arabinose, mannose, malt extract starch hydrolysate, or the like.

As a nitrogen source, similarly, various synthetic or natural materials that can be utilized by the bacterial strain, such as peptone, meat extract, yeast extract, can be utilized.

Further, an inorganic salt such as salt or phosphate, a salt of a metal such as calcium, magnesium, or iron, or a micronutrient source such as a vitamin or an amino acid can also be added where necessary in accordance with a normal method of microbial culture.

Other components contained in the microbial material is not particularly limited as long as they are components generally contained in microbial materials. These components may be, for example, a carrier such as a porous material for adsorbing or stabilizing micro-organisms, various organic materials, fertilizer component, or other minerals used as a nutritious source during micro-organism proliferation, or a diluent, a dispersant, or the like.

As described above, the bacterial strain disclosed in this application has a nitrogen fixing capability and a N₂O reduction capability. Therefore, the microbial material containing the bacterial strain functions as a growth-promoting agent for a plant and also functions as an emission inhibitor for N₂O from a farm field. Plants to promote growth may be a leguminous plant (soybean, peanut, green gram, or the like), a gramineous plant (sorghum, corn, wheat, barley), or the like.

### Embodiment of Plant Cultivation Method

The plant cultivation method disclosed in this application includes steps of causing the above microbial material to be in contact with a seed or a root of a plant or to be present near a root of a plant. This step promotes growth of the plant. Note that, in this specification, “root” means a part that is in the soil or a hydroponic solution to absorb water or nutrition when the plant is cultivated. Further, “promote growth” means to promote growth of a host plant by fixing nitrogen regardless of whether or not there is nodulation.

Although Examples will be presented below and the embodiment disclosed in this application will be specifically described, these Examples are simply provided for the purpose of illustration of the embodiment, which are intended neither to limit the scope of the invention disclosed in the present application nor to express limitation of the same.

### EXAMPLE

### [Isolation of Bacterial Strain]

A bacterial extraction liquid was prepared from surface-sterilized sorghum roots, the extraction liquid was inoculated into soybean seeds, and the Bradyrhizobium bacteria were isolated from the formed root nodules. Specific procedures are as follows.

Sorghum roots of about 30 g harvested from a farm field in Nihonmatsu city, Fukushima prefecture were washed with 70% ethanol and then immersed in 2.5% NaOCl at room temperature for 10 minutes to sterilize the surfaces. After washed with sterilized distilled water for 10 times, the sorghum roots were crushed by using sterilized mortar and pestle while frozen in liquid nitrogen. About 200 mL of a Tris-HCl buffer (50 mM, pH7.5) was added to and sufficiently mixed with the crushed sorghum roots, and plant residuals were removed by filtrating the mixture by using Miracloth (Milipore). The filtrate was centrifuged (at 9.876×g for 10 minutes), and the precipitate was suspended in a Tris-HCl buffer (50 mM, pH7.5), the liquid volume was adjusted to 10 mL. Five surface-sterilized soybean seeds (Glycine max cv. Enrei) were placed apart on a Leonardo Jar (Inaba et al., “N₂O Emission from Degraded Soybean Nodules Depends on Denitrification by Bradyrhizobium japonicum and Other Microbes in the Rhizosphere”, Microbes Environ., 2012, December; 27(4): 470-476), and the bacterial extraction liquid was dropped at 1 mL per seed. Five individuals per pot were seeded and cultivated for three weeks in a growth chamber (Koito Electric Industries, at 23° C., for 16 h light and 8 h dark). The root nodules hosted on the soybeans were then collected. The root nodules were washed with 70% ethanol for 1 minute and then immersed in 0.5% NaOCl for 10 minutes to sterilize the surfaces. The root nodules were cut by a flame-sterilized cutter, and the inside of the root nodule cross section was touched with a sterile toothpick and streaked on a 100-fold diluted NA agar plate culture medium (Difco (registered trademark), Nutrient broth). After cultured at 28° C. for 10 days, emerged colonies were isolated by further performing single-colony purification with the 100-fold diluted NA agar culture medium, and isolated strains are obtained.

Next, the analysis method, the measuring method, and the like used in the Examples will be described.

### [ITS Sequence]

The 16S-23S rRNA intergenic region (ITS) of the isolated strains was amplified by a PCR method, and the base sequence was then determined by a Sanger method. Note that the PCR was performed with the composition of a PCR reaction liquid indicated in Table 1 under reaction conditions indicated in Table 2 by using Blend taq (registered trademark)-plus-(TOYOBO CO., LTD., Okasa) for improving the performance and by using ITS-F and ITS-R described in Saeki et al., “Grouping of Bradyrhizobium USDA Strains by Sequence Analysis of 16S rDNA and 16S-23S rDNA Internal Transcribed Spacer Region”, Soil Sci. Plant Nutr., 50(4), 517-525, 2004 for the primer.

TABLE 1

| Regent | volume | |
|---|---|---|
| 10× Blend taq PCR buffer | 2.5 | μL |
| 2 mM dNTPs | 2.5 | μL |
| Primer (forward) | 0.5 | μL |
| Primer (Reverse) | 0.5 | μL |
| Blend taq plus | 0.25 | μL |
| Templete DNA (Cell lysate) | 1 | μL |
| Water | 17.75 | μL |

TABLE 2

| Step | Tempareture | Time | | Numer of cycle |
|---|---|---|---|---|
| Initial denaturation | 94° C. | 2 | min | 1 |
| Denaturation | 94° C. | 30 | sec | 30 |
| Annealing | 55° C. | 30 | sec | |
| Extention | 72° C. | 1 | min | |

| | | | |
|---|---|---|---|
| Hold | 12° C. | ∞ | 1 |

BLAST search was performed on the determined ITS sequence by NCBI (https://www.ncbi.nlm.nih.gov/), and it was confirmed that SG09 and SG11 obtained in this example belong to the Bradyrhizobium.

### [Evolutionary Phylogenetic Tree by AMPHORA]

The base sequence information of draft genomes of the isolated strains was uploaded to DFAST (https://dfast.nig.ac.jp/) and converted into an amino acid. To analyze a phylogenetic relationship, the 31 amino acid sequences of housekeeping gene described above were extracted by using AMPHORA (Wu and Scott, 2012). The amino acid sequences of the extracted genes are coupled, and a phylogenetic tree was drawn by using MEGA v.7.0 (Sudhir Kumar et al., “MEGA7: Molecular Evolutionary Genetics Analysis Version 7.0 for Bigger Datasets”, Mol. Biol. Evol. 33(7): 1870-1874, 2016) and using the neighbor-joining method (Naruya Saitou et al., “The Neighbor-joining Method: A New Method for Reconstructing Phylogenetic Trees”, Mol. Biol. Evol. 4(4): 406-425, 1987) (bootstrap=1000 times).

### [Phylogenetic Tree of Bradyrhizobium Bacterial Isolated Strain Based on ITS Sequence]

The OTU was created at 97% identity based on the ITS sequence. As a result, it was confirmed that the isolated strains were multiple types of bacterial strains included in the ottawaense Glade, the diazoefficiens Glade, and the japonicum Glade.

### [Measurement of N₂O Reduction Activity]

The N₂O reduction activity was measured by observing the decrease of added N₂O. First, 15 mL of HMM culture medium (Sameshima-Saito et al. 2006) in a 75 mL test tube was inoculated with the target bacterial strain, and the target bacterial strain was pre-cultured at 30° C. under an aerobic condition for about 6 hours. Next, a butyl rubber plug was put on the test tube containing the bacterial liquid, and the gas phase was substituted with 4.98% N₂O gas (95.02% N₂). Culture was made with shaking for 12 to 14 hours to induce N₂O reduction. The culture liquid was mixed with a sterilized medium to adjust the absorbance to about 0.05 (optical path=10 mm). Before this, the culture medium and the gas phase in the test tube to be added were degassed by a N₂ gas in advance in order to maintain an anaerobic condition, and the dead space of plastic syringes and needles to be used was also washed with a N₂ gas for three times. A prepared bacterial liquid of 10 mL was transferred to the sterilized test tube filled with 100% N₂ gas, and the bacterial liquid volume and the gas phase volume were adjusted to the same among the strains. Note that, with the butyl rubber plug put on, the volume of the test tube was 73.8±0.2 mL, and the volume of the gas phase was 63.8 mL. The 100% N₂O gas of 0.65 mL was introduced to the test tube to adjust the final concentration to about 1%. This point of time was defined as 0 hour, and the decrease in the N₂O concentration was measured over time. The N₂O concentration was measured under the condition of Table 3 below by using gas chromatography (Shimadzu, GC2014).

TABLE 3

| GC2014 (Shimazu, Co., Ltd, Kyoto, Japan) | | |
|---|---|---|
| Colum | Porapak Q | |
| Carrier gas | He | |
| Colum temp. | 50° | C. |
| Injector temp. | 100° | C. |
| Detector temp. | 100° | C. |
| Flow | 30 | mL/min |
| Retention time (N₂) | 0.76 | min |
| Retention time (N₂O) | 3.48 | min |
| Retention time (CO₂) | 2.66 | min |

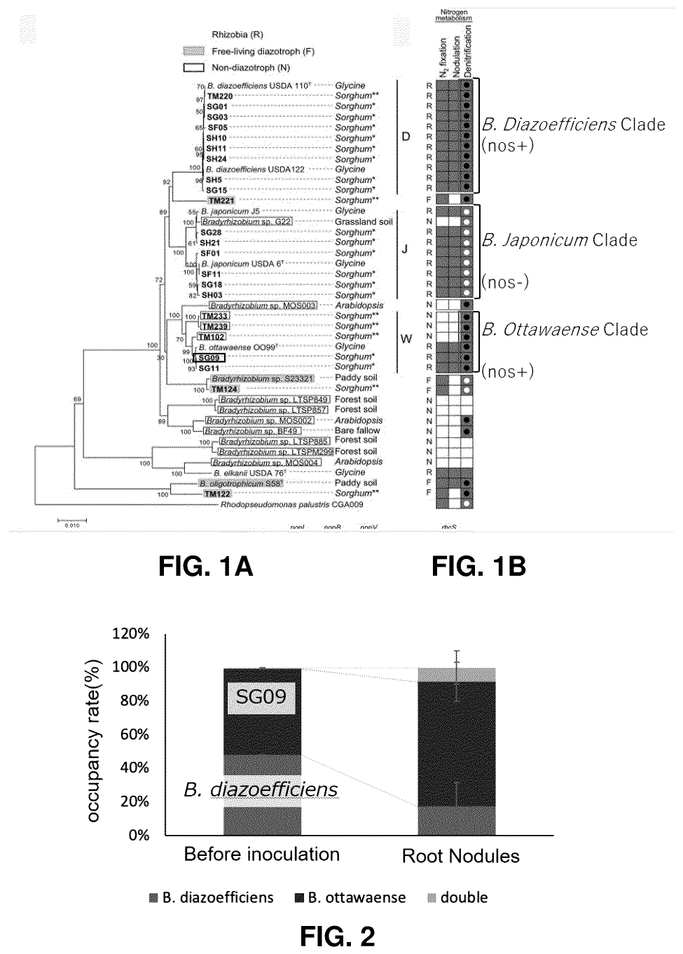

FIG. 1A is a diagram illustrating an evolutionary phylogenetic tree by AMPHORA (housekeeping gene) of Bradyrhizobium bacterial strains, and FIG. 1B is a diagram illustrating the function of each bacterial strain. For N₂ fixation (nitrogen fixation) and Nodulation (nodulation) of FIG. 1B, each black square represents possession of the gene group of interest, and each white square represents no possession of the gene group of interest. Further, for Denitrification (denitrification), each black square represents possession of the gene group of interest, each black circle represents presence of the nos-gene group that is the final process of denitrification (gene group encoding enzyme or the like that reduce N₂O into N₂), and each white circle represents absence of the nos-gene group. Note that, in addition to the SG09 and SG11 obtained in this example, FIG. 1 illustrates known Bradyrhizobium bacterial strains for comparison. Among these strains, the OO99 that is a known soybean rhizobium included in the ottawaense Glade is a type strain that is a reference for the ottawaense species. Note that the details of TM102, TM233, and TM239 included in the ottawaense Glade are described in Shintaro Hara et al., “Identification of Nitrogen-Fixing Bradyrhizobium Associated With Roots of Field-Grown Sorghum by Metagenome and Proteome Analyses”, March 2019|Volume 10|Article 407.

Further, ANI analysis was performed on the full-length genome of the obtained SG09 strain and the full-length genome of the obtained OO99 strain by using a known scheme, which results in that the SG09 strain exhibits an ANI value of 99.08% with respect to the OO99 strain.

From the above results, it was confirmed (1) that the SG09 strain obtained in this example is a bacterial strain that is the same species as but is different bacterial strain from the OO99 that is a known soybean rhizobium included in the ottawaense Glade and (2) that, in the bacterial strains included in the ottawaense Glade, there are a strain having a N₂O reduction capability and a nitrogen fixing capability and a strain having a N₂O reduction capability but no nitrogen fixing capability.

### Production of Microbial Material and Observation of Nodulation

### Example 1

The microbial material was produced by suspending the SG09 strain isolated in [Isolation of Bacterial Strain] described above with sterilized water. Next, a microbial material was produced by mixing the SG09 strain and USDA122 (BCRC number: 13533) belonging to the same cluster in 16S ribosomal RNA gene phylogenetic analysis as B. diazoefficiens USDA110, which is a known soybean rhizobium, at a ratio of 1:1 at a density of 0.5×10⁷ cells/mL. The details of USDA122 are described in Masayuki Sugawara et al., “Complete Genome Sequence of Bradyrhizobium diazoefficiens USDA122, a Nitrogen-Fixing Soybean Symbiont”, Genome Announc. 2017, doi: 10.1128/genome A. 01743-16. Four seeds of soybean (Glycine max, cv. Enteri) were sown into a Leonardo Jar pot into which sterilized vermiculite was put, and inoculated for separate four times with 1 mL of the bacterial solution (microbial material) adjusted above. In this adjustment, the bacterial solution was dropped from a position as far as possible (approximately 1 cm) so as not to directly contact the soybean seed. After the soybeans were germinated, three of four germinated soybeans were removed, and one individual per pot was grown. The cultivation was carried out for 26 days by using a growth chamber (Koito Electric Industries, at 23° C., for 16 h light and 8 h dark).

FIG. 2 is a graph illustrating a survey of the occupancy rate of the formed root nodules. As is clear from FIG. 2, despite the same number of bacterial cells of the inoculated bacterial strains, the dominance of the SG09 in the soybean root nodules on the 26th day was about 74%. From the above result, it was revealed that the SG09 is superior to the B. diazoefficiens USDA122, which is the known soybean rhizobium, in the competitive nodulation capability.

### Observation of Growth-Promoting Capability of SG09

### Example 2

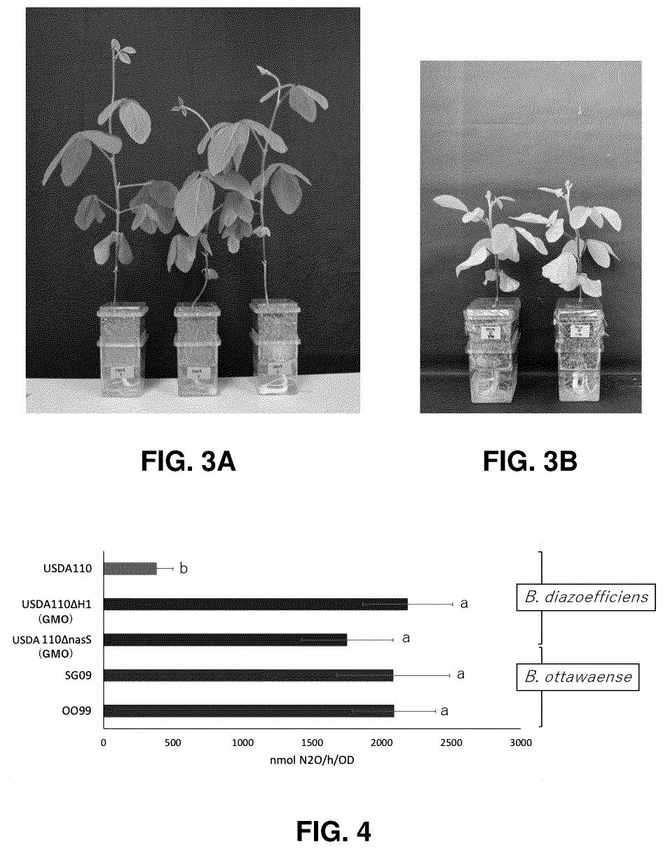

By using microbial materials containing only the SG09 strain produced in Example 1 (the density of SG09 strain: 0.5×10 7 cells/mL), soybeans were grown in the same procedure as in Example 1. FIG. 3A illustrates a photograph of the soybean grown in Example 2 and taken on the 26th day after the inoculation.

### Comparative Example 1

An experiment was performed with the same procedure as that in Example 2 except that the microbial material was not inoculated. FIG. 3B illustrates a photograph of soybeans grown in Comparative example 1.

As is clear from FIG. 3A and FIG. 3B, no sign of nitrogen deficiency or the like was observed in the soybeans in Example 2 inoculated with the SG09. In contrast, the soybeans of Comparative example 1 not inoculated with the SG09 showed worse growth than Example 2, had yellowed lower lobes, and exhibited symptoms of nitrogen deficiency. From the above results, it was confirmed that the SG09 lives symbiotically with soybeans and achieves a normal nitrogen fixing capability despite being isolated from the sorghum root.

### [Comparison of N₂O Reduction Capability]

Next, comparison on the N₂O reduction capability was made between the wild-type strain belonging to the B. ottawaense Glade and a strain belonging to the B. diazoefficiens Glade. FIG. 4 is a graph illustrating the difference in the N₂O reduction capability between wild-type strains belonging to the B. ottawaense Glade (SG09, OO99), a wild-type strain belonging to the B. diazoefficiens Glade (USDA110, JCM number: 10833), and mutant strains with a N₂O reduction capability enhanced by gene manipulation (USDA110ΔH1, USDA110ΔnasS). Note that the USDA110ΔH1 is a mutant strain gene-manipulated in accordance with the procedure described in Non-Patent Literature 1, and the USDA110ΔnasS is a mutant strain gene-manipulated in accordance with the procedure described in Non-Patent Literature 2. The measurements of FIG. 4 are values measured by [Measurement of N₂O Reduction Activity] described above.

As illustrated in FIG. 4, it was confirmed that the SG09 and the OO99 that are wild-type strains belonging to the B. ottawaense Glade have a N₂O reduction capability that is even about 5.4 times higher than that of the USDA110 that is a wild-type strain belonging to the B. diazoefficiens Glade and have substantially the same N₂O reduction capability as USDA110ΔH1 and USDA110ΔnasS that are mutant strains with an enhanced N²O reduction capability. Further, in a significance test performed with Tukey's HSD test, it was confirmed that a and b were significantly different at p<0.05.

[Search for Bacterial Strain Other than SG09 Strain]

Other bacterial strains included in the ottawaense Glade were searched for in the same procedure as in [Isolation of Bacterial Strain], [ITS Sequence], [Evolutionary Phylogenetic Tree by AMPHORA], [Phylogenetic Tree of Bradyrhizobium Bacterial Isolated Strain Based on ITS Sequence], and [Measurement of N₂O Reduction Activity] described above.

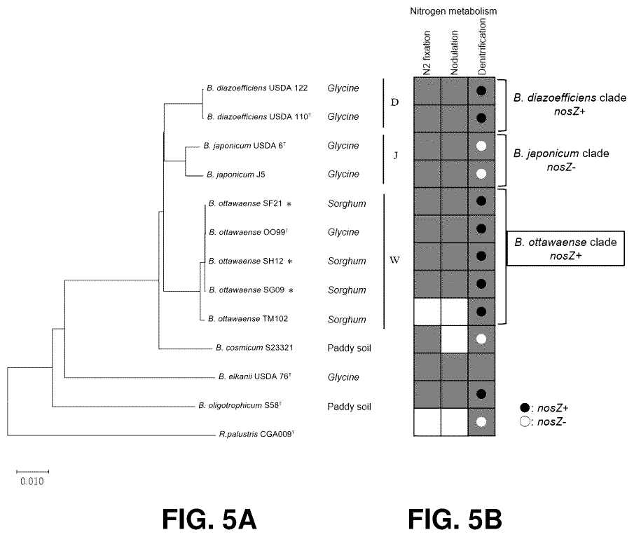

FIG. 5A is a diagram illustrating an evolutionary phylogenetic tree by AMPHORA (housekeeping gene) of a Bradyrhizobium bacterial strain, and FIG. 5B is a diagram illustrating the function of each bacterial strain. Note that description for the symbols (black square, white square, black circle, white circle) in the fields of “N₂ fixation”, “Nodulation”, and “Denitrification” in FIG. 5B is the same as that in FIG. 1B.

Further, ANI analysis was performed on the full-length genomes of the obtained SF21 strain and the SH12 strain and the full-length genome of the OO99 strain by using a known scheme, which results in that the SF21 strain exhibits an ANI value of 99.1% with respect to the OO99 strain, and the SH12 strain exhibits an ANI value of 99.1% with respect to the OO99 strain.

From the above results, it was confirmed that the newly obtained SF21 strain and SH12 strain are the same type as OO99, which is a known soybean rhizobium included in the ottawaense Glade, but are different bacterial strains.

### [Production of Microbial Material and Observation of Growth-Promoting Capability of the Same]

### Example 3

The microbial material was produced by suspending the isolated SG09 strain, SF21 strain, and SH12 strain in sterilized water to have a density of 1×10⁹ cells/mL, respectively. Five seeds per pot of soybeans (Glycine max, cv. Enteri) whose surfaces were sterilized by 0.5% sodium hypochlorite were sown into a Leonardo Jar pot into which sterilized vermiculite was put, and these five seeds were inoculated with 1 ml of the microbial material. The cultivation was carried out by using a growth chamber under conditions of at 25° C. and 16 hours for a light period/8 hours for a dark period, reduction was performed leaving three individuals which were in a good germination state on the third day after the sowing, and cultivation was carried out for another 27 days. A nitrogen-free hydroponic solution was periodically supplied to the pot. After the cultivation, root nodules were harvested on a pot basis. After the number of root nodules was measured on a pot basis, the root nodules were dried at 80° C. for 48 hours, and the dry weight thereof was measured immediately after the drying.

### Comparative Example 2

An experiment was performed with the same procedure as that in Example 3 except that B. diazoefficiens USDA110 (JCM number: 10833) was used as a bacterial strain.

### Comparative Example 3

An experiment was performed with the same procedure as that in Example 3 except that the microbial material was not inoculated.

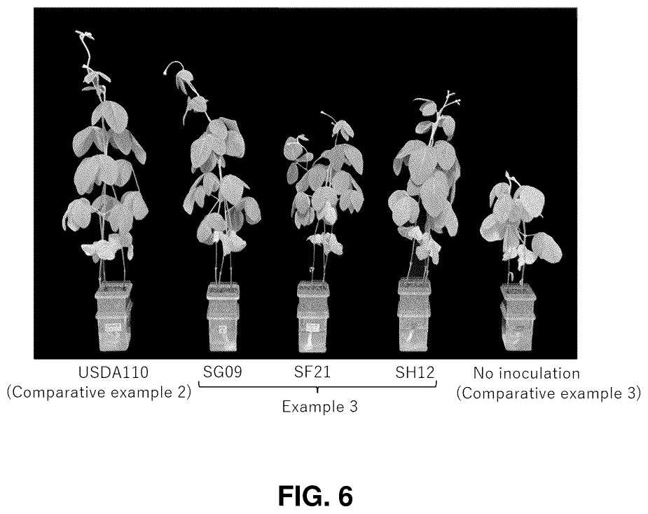

FIG. 6 illustrates a photograph of soybeans grown in Example 3, Comparative example 2, and Comparative example 3. As is clear from FIG. 6, the growth condition of the soybeans of Example 3, which were inoculated with the microbial material, is slightly poorer than that of Comparative example 2 inoculated with the microbial material using the known soybean rhizobium but is better than that of Comparative example 3 on which no inoculation of the microbial material was performed.

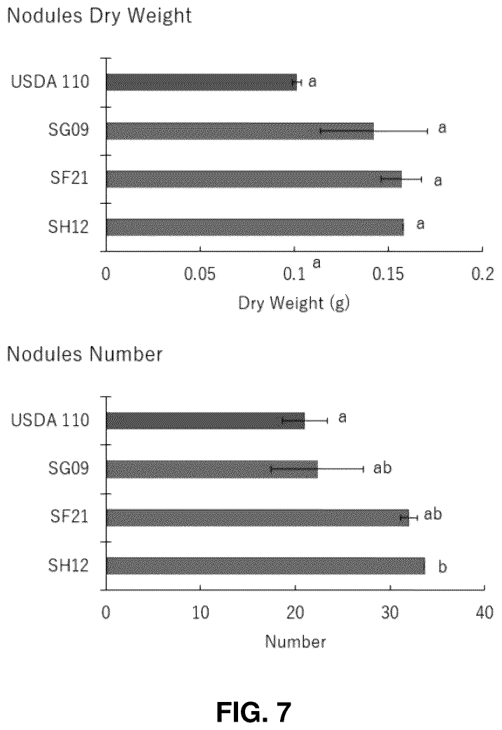

FIG. 7 illustrates the dry weight of the root nodules (Nodules Dry Weight) of soybeans grown in Example 3 and Comparative example 2 and the number of root nodules (Nodules Number) of soybeans grown in Example 3 and Comparative example 2. As is clear from FIG. 7, it was confirmed that the use of the SG09 strain, the SF21 strain, and the SH12 strain as the microbial material increases the number of root nodules and the volume of a root nodule compared to Comparative example 2 (USDA110).

[Comparison of N₂O Reduction Capability between SF21 and SH12]

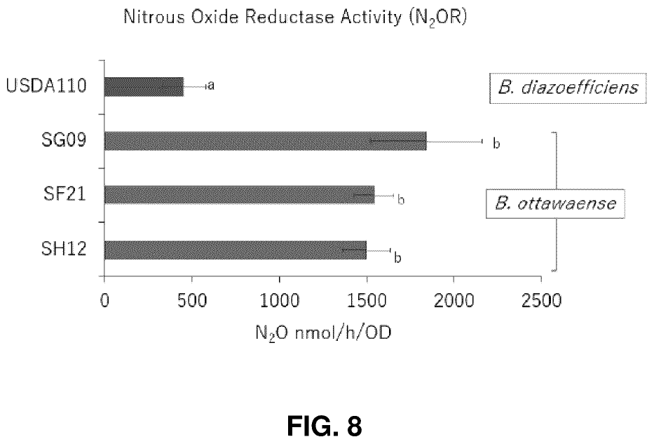

Next, the USDA110, the SG09, the SF21, and the SH12 were used as bacterial strains to compare N₂O reduction capabilities in the same procedure as in [Comparison of N₂O Reduction Capability] described above. FIG. 8 aillustrates the results thereof. As illustrated in FIG. 8, it was confirmed that each of the SG09, the SF21, and the SH12 that are wild-type strains belonging to the B. ottawaense Glade has a higher N₂O reduction capability than USDA110 that is a wild-type strain belonging to the B. diazoefficiens Glade. Further, in a significance test performed with Tukey's HSD test, it was confirmed that a and b were significantly different at p<0.05.

### [Aged Root Nodule N₂O Flux Measurement]

### Example 4

Next, the aged root nodule N₂O flux of the SG09 strain was measured. The procedure will be illustrated below.

### Soybean Cultivation

The microbial material was produced by suspending the isolated SG09 strain in sterilized water to have a density of 1×10⁸ cells/mL. Soybean seeds (Glycine max, cv. Enteri) whose surfaces were sterilized by 0.5% sodium hypochlorite were sown into a Leonardo Jar pot into which sterilized vermiculite was put at three seeds per pot, and were inoculated with 1 ml of the microbial material. The cultivation was carried out by using a growth chamber under conditions of 25° C. and 16 hours for a light period/8 hours for a dark period, reduction was performed leaving one individual which was in a good germination state on the third day after the sowing, and cultivation was carried out for another 27 days. A nitrogen-free hydroponic solution was periodically supplied to the pot.

### Root Nodule Aging Treatment

For root nodule aging treatment, soil from Tohoku University Kashimadai Farm Field was used. The soil was sieved with a sieve of 2 mm, and portions of soil were put in 50 ml centrifugal tubes where each portion was 10 g and the number of centrifugal tubes was the same as the number of pots. To wash the soil, 30 ml of distilled water was added to the centrifugal tubes, which was shaken for 10 minutes and then centrifuged at 5000 g for 10 minutes, and the supernatant was discarded. This was repeated three times, 30 ml of distilled water was then added to the centrifugal tube, and the soil was well suspended. To artificially promote the aging of the soybean root nodules, parts grown above the soil level were cut out, and 10 g of soil suspended in 30 ml of distilled water was added to the pot in which the root system (including root nodules) remained. And then pots which are added soil suspension were allowed under conditions of 25° C. and 16 hours for a light period/8 hours for a dark period for 20 days to promote aging of the root nodules.

### N₂O Flux Measurement

Root systems were harvested from the pots and put into a glass vial having a volume of 60 mL, the glass vial was sealed, and the root systems were incubated at 25° C. for 3 hours. Gas-phase sampling was performed before and after the incubation, ECD gas chromatograph (Shimadzu, G-C2014) was used to measure the N₂O concentration in the gas phase, and N₂O flux under the atmospheric condition was calculated.

### Comparative Example 4

The aged root nodule N₂O flux measurement was performed in the same procedure as in Example 4 except that the USDA110 strain was used instead of the SG09 strain.

### Comparative Example 5

The aged root nodule N₂O flux measurement was performed in the same procedure as in Example 4 except that the USDA110ΔH1 strain was used instead of the SG09 strain.

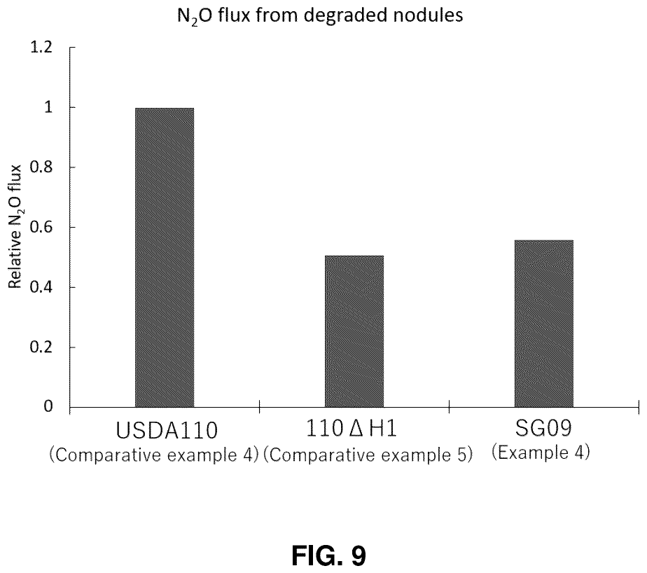

FIG. 9 is a graph illustrating N₂O flux measurements of Example 4 and Comparative example 5 relatively when the N₂O flux measurement of the USDA110 strain of Comparative example 4 (the wild-type strain of B. diazoefficiens that is a known soybean rhizobium) is defined as 1. As is clear from FIG. 9, it was confirmed that the SG09 strain (Example 4), which is a wild-type strain, can suppress by 50% of N₂O flux of the USDA110, which is a wild-type strain, and can suppress the N₂O flux to substantially the same level as Comparative example 5, which is a mutant strain (USDA110ΔH1) whose N₂O reduction capability was enhanced by genetic manipulation.

From the above results, it was confirmed about the bacterial strains belonging to the B. ottawaense Glade (1) that these bacterial strains have a higher N₂O reduction capability even in wild-type strains, (2) that there are a strain having a nitrogen fixing capability and a strain having no nitrogen fixing capability, and (3) that the strain having the nitrogen fixing capability lives symbiotically with soybeans and promote growth thereof. Therefore, the use of the microbial material containing a bacterial strain belonging to the B. ottawaense Glade having a nitrogen fixing capability and a N₂O reduction capability in a farm field achieves advantageous effects of enabling both promotion of growth of a plant and suppression of N₂O emission to the atmosphere.

### INDUSTRIAL APPLICABILITY

The use of the microbial material disclosed in this application can promote growth of plants and suppress N₂O emission to the atmosphere. Therefore, the microbial material disclosed in this application is useful in the agricultural field.

1. A microbial material comprising a bacterial strain belonging to a Bradyrhizobium ottawaense Glade having a nitrogen fixing capability and an N₂O reduction capability.

2. The microbial material according to claim 1, wherein the bacterial strain belongs to a Glade including a B. ottawaense type strain OO99 in evolutionary phylogenetic analysis in which the B. ottawaense type strain OO99 and one or more Bradyrhizobium distinct species thereof are included in an operational taxonomic unit (OTU).

3. The microbial material according to claim 2, wherein the evolutionary phylogenetic analysis creates and analyzes, for each OTU, a linked sequence in which amino acid sequences are linked to each other, the amino acid sequences being encoded by genes of dnaG, frr, infC, nusA, pgk, pyrG, rplA, rplB, rplC, rplD, rplE, rplF, rplK, rplL, rplM, rplN, rplP, rplS, rplT, rpmA, rpoB, rpsB, rpsC, rpsE, rpsI, rpsJ, rpsK, rpsM, rpsS, smpB, and tsf extracted by using AMPHORA.

4. The microbial material according to claim 1, wherein the bacterial strain further has an ANI value for the B. ottawaense type strain OO99 that is 95% or higher in ANI analysis.

5. The microbial material according to claim 1, wherein the bacterial strain further has an ITS base sequence having 97% or higher homology with an ITS (16S-23S rRNA intergenic region) base sequence of a Bradyrhizobium bacterium.

6. The microbial material according to claim 1, wherein the bacterial strain is SG09 (accession number: NITE BP-03361).

7. The microbial material according to claim 1, wherein the bacterial strain is SF21 (accession number: NITE BP-03552) or SH12 (accession number: NITE BP-03553).

8. The microbial material according to claim 1 that functions as a growth-promoting agent for a plant.

9. The microbial material according to claim 8, wherein the plant is a leguminous plant.

10. A plant cultivation method comprising a step of causing the microbial material according to claim 1 to be in contact with a seed or a root of a plant or to be present near a root of a plant.

11. A bacterial strain that belongs to a Bradyrhizobium ottawaense Glade having a nitrogen fixing capability and an N₂O reduction capability and belongs to a Glade including a B. ottawaense type strain OO99 in evolutionary phylogenetic analysis in which the B. ottawaense type strain OO99 and one or more Bradyrhizobium distinct species thereof are included in an operational taxonomic unit (OTU).

12. The bacterial strain according to claim 11, wherein the bacterial strain is SG09 (accession number: NITE BP-03361).

13. The bacterial strain according to claim 11, wherein the bacterial strain is SF21 (accession number: NITE BP-03552) or SH12 (accession number: NITE BP-03553).

14. The microbial material according to claim 2, wherein the bacterial strain further has an ANI value for the B. ottawaense type strain OO99 that is 95% or higher in ANI analysis.

15. The microbial material according to claim 2, wherein the bacterial strain further has an ITS base sequence having 97% or higher homology with an ITS (16S-23S rRNA intergenic region) base sequence of a Bradyrhizobium bacterium.

16. The microbial material according to claim 4, wherein the bacterial strain further has an ITS base sequence having 97% or higher homology with an ITS (16S-23S rRNA intergenic region) base sequence of a Bradyrhizobium bacterium.

17. The microbial material according to claim 14, wherein the bacterial strain further has an ITS base sequence having 97% or higher homology with an ITS (16S-23S rRNA intergenic region) base sequence of a Bradyrhizobium bacterium.

18. The microbial material according to claim 2, wherein the bacterial strain is SG09 (accession number: NITE BP-03361).

19. The microbial material according to claim 4, wherein the bacterial strain is SG09 (accession number: NITE BP-03361).

20. The microbial material according to claim 5, wherein the bacterial strain is SG09 (accession number: NITE BP-03361).
